(19) United States
(12) Patent Application Publication
(10) Pub. No.: US 2021/0015467 A1
(43) Pub. Date: Jan. 21, 2021

(54) URINE COLLECTION ASSEMBLY

(71) Applicant: Deborah GLASS, Los Angeles, CA (US)

(72) Inventor: Deborah GLASS, Los Angeles, CA (US)

(21) Appl. No.: 16/924,565

(22) Filed: Jul. 9, 2020

Related U.S. Application Data
(60) Provisional application No. 62/921,948, filed on Jul. 17, 2019.

(51) Int. Cl. A61B 10/00

(52) U.S. Cl. CPC A61B 10/007; A61B 10/0038

(57) ABSTRACT

A urine collection container assembly with a main container, a container cap, a flexible funnel, a double-sided adhesive ring panel and a plurality of double-sided adhesive attachment panels. The main container has a flat upper ring surface and a side skirt having threads. The container cap has female threads that fit the container threads. The flexible funnel is constructed of very thin-wall plastic sheet material having a centrally die cut aperture that matches the outer diameter of the upper perimeter ring of the main container. The adhesive ring panel attaches to the perimeter of the main container and to the underside of the aperture of the funnel member. Additional double-sided adhesive panels attach the flexible funnel member to the top of a standard toilet seat.

### CROSS REFERENCE TO RELATED APPLICATIONS

This application claims the benefit of U.S. provisional application No. 62/921,948 filed on Jul. 17, 2019 and entitled, “Urine Collection Container Assembly.” The contents of the provisional application are incorporated by reference in their entirety herein.

### TECHNICAL FIELD

This invention disclosed herein relates generally to the field of collection containers and more specifically to urine collection containers.

### BACKGROUND

Urine collection containers are generally cylindrical containers, approximately two and one quarter inches tall and approximately two and one quarter inches in diameter with a threaded cap. There is usually a graphic panel on the side of the container to write the name of the user and the date of use. Urine collection containers are generally manufactured under clean room conditions to avoid any contaminates that may affect the urine sample. Sterile urine collection containers usually have a breakable seal to indicate whether the container has been opened.

There is a fundamental issue when females, young children, the elderly, and those with disabilities use these urine collection containers because guiding a stream of urine into a collection container cannot be guaranteed. Therefore, there is a need for a special collection container assembly that is more suited to the needs of females, young children, the elderly, and those with disabilities.

### SUMMARY

The following simplified summary provides a basic understanding of some aspects of the claimed subject matter. This summary is not an extensive overview and is not intended to identify key/critical elements or to delineate the scope of the claimed subject matter. Its purpose is to present some concepts in a simplified form as a prelude to the more detailed description that is presented below.

One object of the invention is to provide a urine collection container assembly that allows a person to easily obtain a urine sample in a container. Another object of the invention is to provide a urine collection container assembly that allows a person to sit on a standard toilet seat, deposit the urine sample into a specially formed container, and then easily retrieve the contained sample for testing. Another object of the invention is to provide an excrement collection container assembly. Other objects and advantages of the present invention will become apparent from the following descriptions, taken in connection with the accompanying drawings, wherein, by way of illustration and example, an embodiment of the present invention is disclosed.

In one embodiment is a funnel-shaped member for use in collecting a urine sample. The funnel-shaped member has a narrow end and a wide end. Generally, the funnel-shaped member is made from a flexible material such as plastic. Oftentimes, the funnel-shaped member is sterile. In some embodiments, the funnel-shaped member is specifically designed to attach to a urine collection container. Generally, the narrower end of the funnel-shaped member attaches to the urine collection container. Various attachment methods can be used, including, but not limited to adhesive, double sided tape, threading, male to female lock system, and the like. In other embodiments, the funnel-shaped member is designed to be used with a standard toilet seat. In these embodiments, the wide end of the funnel-shaped member has a flange that rests on the rim of the toilet or on the seat of the toilet. In other embodiments, the funnel-shaped member is designed to be hand-held. In these embodiments, the funnel-shaped member has a flange such that the wide end can be used as a handle. In some embodiments, the funnel-shaped member is packaged to maintain sterility. In other embodiments, the funnel-shaped member is packaged with the urine collection container. Optionally, the funnel-shaped member can be packaged by storing the funnel-shaped member inside the urine collection container. In this embodiment, the funnel-shaped member may be pre-attached to the urine collection container.

In one embodiment, there is disclosed a urine collection container assembly comprising: a main container housing, a container cap, and a flexible funnel member. In this embodiment, the main container housing has an upper perimeter ring surface transitioning into a downwardly disposed shoulder having outwardly disposed male threads. The container cap has female threads sized to threadably engage the outwardly disposed male threads of the main container housing. A die cut ring panel can be used to attach the flexible funnel-shaped member to the main container housing. The ring panel optionally has a tab to assist with removal of the flexible funnel-shaped member post urine sample collection.

The flexible funnel member may be constructed of very thin plastic sheet or other water-resistant material (e.g. wax paper, parchment paper, latex, silicone, nitrile, rubber, etc.). The funnel has a centrally die cut aperture that matches the outer diameter of the flat upper ring of the main container housing. The funnel also has one or more fluid overflow slits located along side of the centrally die cut aperture. Using double-sided adhesive, the die-cut ring panel attached on one side to the upper flat perimeter ring of the main container housing and on the other side to the underside of the centrally die cut aperture of the flexible funnel member. One or more attachment panels may be attached to the flexible funnel member to hold the funnel to the top of a standard toilet seat. The attachment panels can be double-sided adhesive. One or more graphic panels may be attached to the container housing or cap. The graphic panels may include instruction on using the collection container assembly. The graphic panels may also be used to record patient information and specimen collection date. After the urine specimen is collected, the main container housing is capable of being removed from the flexible funnel member via the tab attached to the ring panel and the container cap secured to the main container housing to retain the urine sample until the urine needs to be accessed from the main container for testing.

### BRIEF DESCRIPTION OF THE DRAWINGS

The drawings constitute a part of this specification and include exemplary embodiments of the invention, which may be embodied in various forms. It is to be understood that in some instances various aspects of the invention may be shown exaggerated or enlarged to facilitate an understanding of the invention.

FIG. 1 is a perspective view of a urine collection container with the top cap in place.

FIG. 2 is a perspective view of a urine collection container with the top cap removed.

FIG. 3 is a top perspective view of the funnel-shaped member.

FIG. 4 is a bottom perspective view of the funnel-shaped member with the urine collection container attached.

FIG. 5 is a perspective view of the urine collection container assembly mounted onto a standard toilet seat.

FIG. 6 is a perspective view of the urine collection container assembly being removed from the toilet seat.

FIG. 7 is an exploded view of the urine collection container assembly.

FIG. 8 is a perspective view of an embodiment the urine collection container assembly.

FIG. 9 is a is a cross-sectional view of an embodiment of the urine collection container assembly.

### DETAILED DESCRIPTION

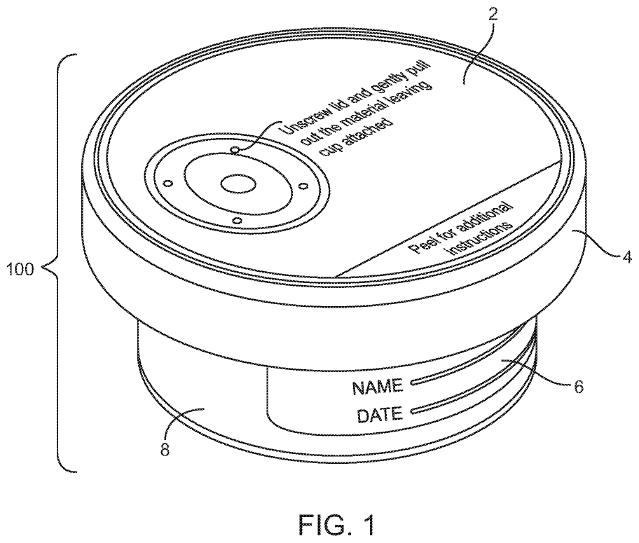

Referring now to FIG. 1 we see a perspective view of the urine collection container assembly 100. A cap, or otherwise described as a lid, 4 is threadably attached to a main container housing 8 which will be hereafter referred to as a cup 8 into which the urine sample is collected. Optionally, the top of the lid 4 includes instructions 2 showing how to use the urine collection container assembly of the present invention. The cup 8 may also include a side graphic 6 intended to be filled in by the user to indicate name of the user and current date of use. In some embodiments, both the lid 4 and the cup 8 can be made from plastic that has been manufactured in a clean room environment. The sterile lids 4 and cups 8 can include a breakable seal to indicate whether the container has been opened.

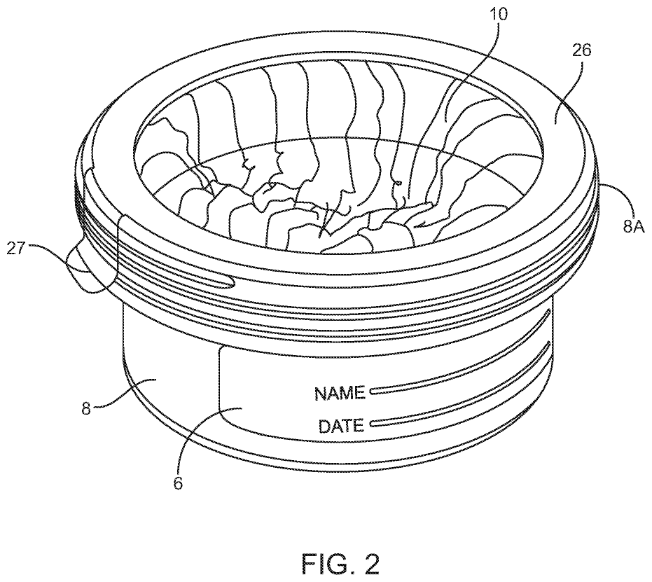

FIG. 2 is a perspective view of the cup 8 with the lid 4 removed. A thin flexible sheet 10 is folded into the inside of the cup 8 and attached to the top perimeter 8A of the cup 8. Non-limiting methods of attachment include adhesive, threading, luer lock, male/female lock system, and the like. When using an adhesive, often the adhesive is one that is removable similar to painter tape or masking tape. Additionally, the adhesive is usually double-sided. In the embodiment illustrated, sheet 10 is attached via a double-sided adhesive ring 26. The adhesive ring 26 includes a tab 27 which can be used to remove the adhesive ring 26 from the cup 8 when needed as will be described in detail below.

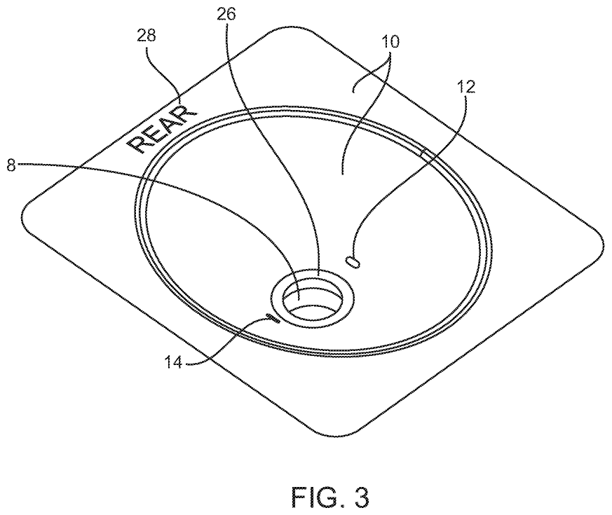

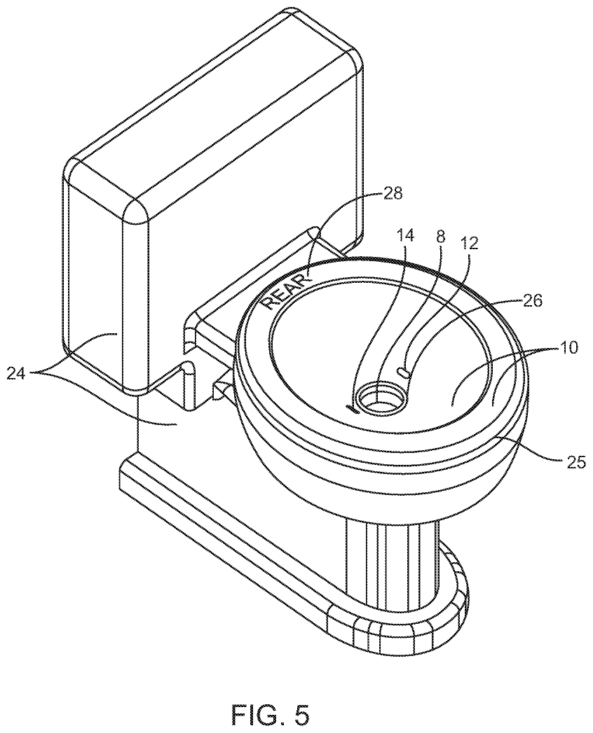

FIG. 3 is a perspective view showing the flexible sheet 10 fully unfurled forming a funnel member and ready for use. In general, sheet 10 is sized to fit standard toilet seats, both round and elongated. In the embodiment illustrated, sheet 10 is shown as a rectangular shape approximately twenty inches long and sixteen inches wide; however, it can also be other shapes such as a circular sheet approximately twenty inches in diameter. Sheet 10 can be made from any thin, flexible, and water-resistant material. Non-limiting materials include wax paper, parchment paper, latex, silicone, nitrile, rubber, plastic, bio-degradable plastics, polymers, and the like. In some embodiments, sheet 10 is 1 mil to 10 mil; is 1 mil to 5 mil; 1 mil to 3 mil thick (1 mil equals one-thousandth of an inch, or 0.001 inch). In some embodiments, sheet 10 is made from two mil low-density polyethylene sheet. In other embodiments, sheet 10 is made from biodegradable vegetable plastic. In one embodiment, cup 8 is approximately three and one eighth inches in diameter, one- and one-half inches deep and is supported by the funnel member sheet 10. The weight of the cup 8 causes the sheet 10 to form a funnel shape. One or more slits 12, 14 are approximately one to two inches long and allow excess liquid to exit the funnel sheet 10 after the cup 8 has been filled with a urine sample. In some embodiments, a graphic printed indicator 28 shows the user that the sheet is to be oriented so that the “rear” of the sheet corresponds to the rear of a standard toilet seat as shown in FIG. 5. The funnel sheet 10 can also made under clean room conditions. The sterile sheets can be packaged with a breakable seal to indicate when the package has been opened.

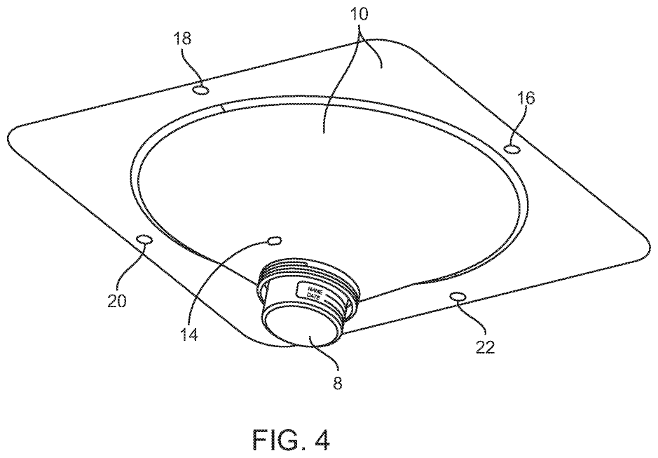

FIG. 4 is a perspective view of the invention showing the underside of funnel sheet 10. One or more adhesive panel members 16, 18, 20, 22 can be included to attach sheet 10 to the toilet seat. Often the adhesive panel members are double-sided adhesive and include a protective layer that is removed to expose the sticky surface prior to attachment to a standard toilet seat.

FIG. 5 is a perspective view showing the urine collection container assembly 100 mounted to a standard toilet seat 25 of a standard toilet 24. When attached as shown, the invention 100 can be used by a person when depositing a urine sample. The person urinates in a normal fashion and the urine travels down the side walls of the funnel sheet 10 and lands in cup 8. Excess urine can exit slits 12, 14.

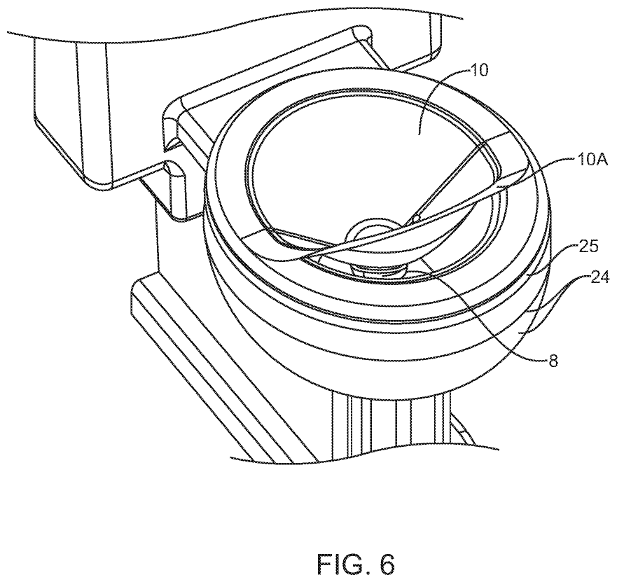

FIG. 6 is a perspective view of the invention where, in a circular version of funnel 10, the forward portion 10A of sheet 10 has been lifted by the user giving access to the cup 8 suspended below the sheet 10. The user then has the ability to grab the cup 8 with one hand and lift and remove the cup 8 and sheet 10 from the toilet seat 25 from the toilet 24. The user can then remove the sheet 10 from the cup 8 by overcoming the adhesive quality of adhesive ring 26, and then dispose of the sheet 10, screw the lid 4 onto cup 8 and write name and date 6 on the side of the cup 8 so that the urine container can be transported to a location where the urine can be tested.

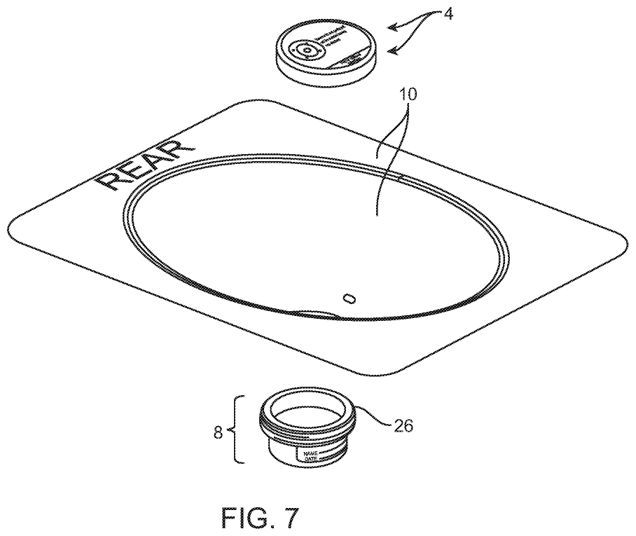

FIG. 7 is an exploded view of the invention where adhesive ring 26 and sheet 10 have been removed from cup 8. Although the funnel member 10 is shown as a rectangular configuration approximately twenty inches long and sixteen inches wide, it can also be made as a circular sheet approximately twenty inches in diameter.

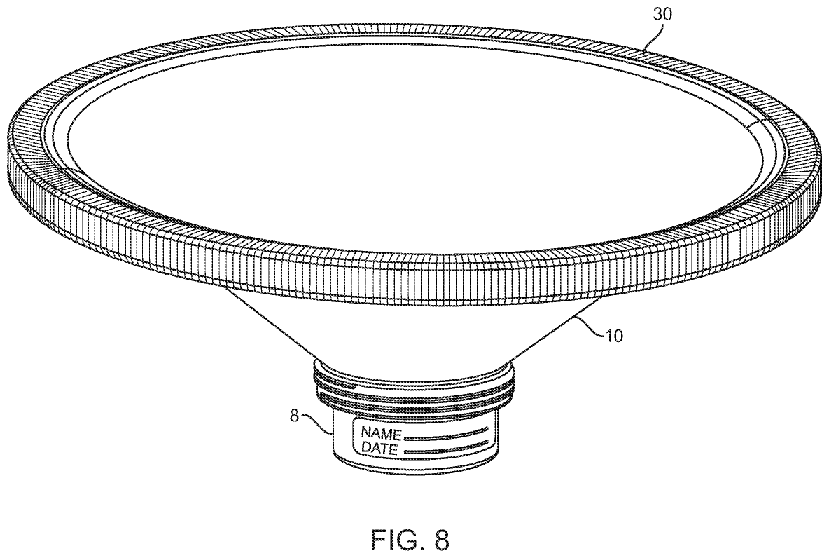

FIG. 8 is a perspective view of the invention illustrating an alternate embodiment for attaching the urine collection assembly to the toilet seat. In this embodiment, sheet 10 has an elastic edge 30. The user stretches elastic edge 30 around and/or over the outer edge of the toilet seat such that the elastic edge helps keep sheet 10 in place during use. Often the elastic edge is a strip or string of elastic material that is ⅛ of an inch wide or less or 1/16 of an inch wide or less or 1/32 of an inch wide or less. In some embodiments, a tab (not shown) may be placed along elastic edge 30 to assist with removal of the urine collection assembly from the toilet.

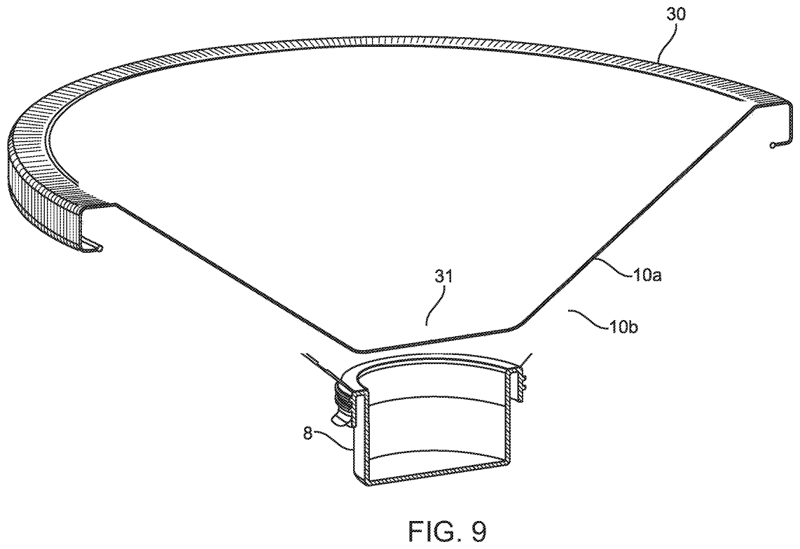

FIG. 9 is a cross-sectional view of the invention illustrating a clean catch embodiment. Clean catch urine specimens involve the user releasing an amount of urine before catching a sample of the urine in the collection cup. In this embodiment, a double layer funnel sheet 10a and 10b is attached to the collection cup. An absorbent pad 31 is placed on sheet 10a. Absorbent pad 31 absorbs the initial flow of urine. After the initial flow of urine, the urine sample is then collected in cup 8. In some embodiments, sheet 10a is designed to open or split apart after the initial urine flow is absorbed into pad 31 thereby allowing continued urine flow to be collected in cup 8. This can be achieved by perforating the pad 31 and/or sheet 10a such that the weight of excess urine creates an opening in sheet 10a that allows urine to then pass into cup 8. In other embodiments, sheet 10a is removed by the user after the initial urine flow is absorbed into pad 31.

Another embodiment, that is not illustrated, is sheet 10 packaged separately from cup 8 and lid 4. Often sheet 10 is packaged with an attachment mechanism that is used to attach sheet 10 to cup 8. In some embodiments, the attachment mechanism is adhesive ring 26. In other embodiments, the attachment mechanism is a ring that can threadably connect with cup 8. In some embodiment, sheet 10 is pre-attached to the attachment mechanism. In some embodiments, sheet 10 includes one or more adhesive panel members 16, 18, 20, 22. In some embodiments, the collection sheet 10 and the attachment mechanism are made under clean room conditions. The packaging can include a breakable seal to indicate that a package has been opened. In these embodiments, the user removes the lid from a urine collection cup, attaches the attachment mechanism to the urine collection cup, unfurls sheet 10 and attaches to a toilet seat using the adhesive panel members.

It is also contemplated that the urine collection assembly can be used to collect fecal samples and may be particularly useful in collecting loose fecal samples.

The urine collection assembly is also contemplated to be used by humans to collect urine and/or fecal samples from animals.

The urine collection assembly is also contemplated to be used on non-toilet facilities such as a bedpan.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

Detailed descriptions of the preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

Certain embodiments of the disclosure are as follows:

1. A collection container assembly comprising: a main container housing; a container cap; a flexible funnel member; a semi-adhesive double-sided die cut ring panel; a graphic cap panel; a plurality of double-sided adhesive attachment panels; a graphic container side panel; the main container housing having a flat upper perimeter ring surface of approximately three hundred thousandths of an inch transitioning into a downwardly disposed shoulder having outwardly disposed male threads; the container cap having female threads sized to threadably engage the outwardly disposed male threads of the main container housing; the die cut ring panel having an integrally attached outwardly disposed tab that does not include adhesive; the flexible funnel member being constructed of very thin-wall plastic sheet material having a centrally die cut aperture that matches the outer diameter of the flat upper perimeter ring of the main container housing as well as one or more fluid overflow slits located along side of the centrally die cut aperture; the semi-adhesive double-sided die cut ring panel attached on one side to the upper flat perimeter ring of the main container housing and on the other side to the underside of the centrally die cut aperture of the flexible funnel member; the plurality of double-sided adhesive attachment panels attached to the underside of the flexible funnel member in a location that corresponds to the top of a standard toilet seat; the graphic cap panel attached to the top surface of the container cap for the purpose of graphically explaining the intended use of the urine collection container; and the graphic container side panel including areas for name and date to identify the user of the urine collection container; the main container housing capable of being removed from the flexible funnel member via the outwardly disposed tab after a urine sample has been deposited and then the container cap secured to the main container housing to retain the urine sample until the urine is accessed from the main container for testing or other purposes.

2. A urine collection container assembly as claimed in claim 1 wherein the main container housing is made of injection molded plastic manufactured in clean room conditions.

3. A urine collection container assembly as claimed in claim 1 wherein the thin sheet funnel material is made of two mil low density polyethylene sheet manufactured in clean room conditions.

4. A urine collection container assembly as claimed in claim 1 wherein the diameter of the main container housing is approximately three and one-eighth inches wide and one- and one-half inches deep.

5. A urine collection container assembly as claimed in claim 1 wherein the flexible funnel member is a flexible rectangular sheet approximately sixteen inches wide and twenty inches long.

6. A urine collection container assembly as claimed in claim 1 wherein the length of each the flexible funnel member slits is approximately one to two inches.

1. A funnel for use with a sample collection container comprising a sheet, a first attachment mechanism, and a second attachment mechanism;
wherein the sheet has an outer edge and a hole in the center creating an inner edge, the hole being similar in size to an opening in the collection container;
wherein the sheet has at least one slit located near the inner edge;
wherein the sheet is made from a thin, flexible, water-resistant material and is sized to fit a toilet seat;
wherein the first attachment mechanism attaches the inner edge to the opening of the collection container; and
wherein the second attachment mechanism secures the outer edge of the sheet to a toilet seat or a bedpan.

2. The funnel of claim 1, wherein the sheet is made from wax paper, parchment paper, latex, silicone, nitrile, rubber, plastic, bio-degradable plastic or polymer.

3. The funnel of claim 1, wherein the sheet is 1 mil to 5 mil thick.

4. The funnel of claim 1, wherein the at least one slit is one to two inches in length.

5. A funnel for use with a sample collection container comprising a first sheet, a second sheet, an adsorbent pad, a first attachment mechanism, and a second attachment mechanism;
wherein the first sheet has an outer edge and the adsorbent pad in about the center;
wherein the second sheet has an outer edge and a hole in the center creating an inner edge, the hole being similar in size to an opening in the collection container;
wherein the second sheet has at least one slit located near the inner edge;
wherein the first sheet is stacked on top of the second sheet;
wherein the first sheet and the second sheet are made from a thin, flexible, water-resistant material and are sized to fit a toilet seat or bedpan;
wherein the first attachment mechanism attaches the inner edge of the second sheet to the opening of the collection container; and
wherein the second attachment mechanism secures the outer edge of the first sheet and the outer edge of the second sheet to a toilet seat or a bedpan.

6. The funnel of claim 5, wherein the first sheet and the second sheet are made from wax paper, parchment paper, latex, silicone, nitrile, rubber, plastic, bio-degradable plastic or polymer.

7. The funnel of claim 5, wherein the first sheet and the second sheet are 1 mil to 5 mil thick.

8. The funnel of claim 5, wherein the at least one slit in the second sheet is one to two inches in length.

9. The funnel of claim 5, wherein the first attachment mechanism is adhesive, threading, luer lock, or male/female lock.

10. The funnel of claim 1, wherein the first attachment mechanism is adhesive, threading, luer lock, or male/female lock.

11. The funnel of claim 1, wherein the second attachment mechanism is adhesive or elastic.

12. The funnel of claim 1, wherein the funnel is manufactured in clean room conditions.

13. A sample collection assembly comprising: a collection container, the collection container having an opening, and a lid that covers the opening; and the funnel of claim 1; wherein the funnel is stored inside the container prior to use.

14. The assembly of claim 13, wherein the sheet is made from wax paper, parchment paper, latex, silicone, nitrile, rubber, plastic, bio-degradable plastic or polymer.

15. The assembly of claim 13, wherein the sheet is 1 mil to 5 mil thick.

16. The assembly of claim 13, wherein the at least one slit is one to two inches in length.

17. A sample collection assembly comprising: a collection container, the collection container having an opening, and a lid that covers the opening; and the funnel of claim 5; wherein the funnel is stored inside the container prior to use.

18. The assembly of claim 17, wherein the first sheet and the second sheet are made from wax paper, parchment paper, latex, silicone, nitrile, rubber, plastic, bio-degradable plastic or polymer.

19. The assembly of claim 17, wherein the first sheet and the second sheet are 1 mil to 5 mil thick.

20. The assembly of claim 17, wherein the at least one slit in the second sheet is one to two inches in length.

21. The assembly of claim 13, wherein the first attachment mechanism is adhesive, threading, luer lock, or male/female lock.

22. The assembly of claim 13, wherein the second attachment mechanism is adhesive or elastic.

23. The assembly of claim 13, wherein the collection container, funnel, first attachment mechanism, and second attachment mechanism are manufactured under clean room conditions.

24. The sample collection assembly of claim 13, wherein the collection container is made of injection molded plastic manufactured in clean room conditions.

25. The sample collection assembly of claim 13, wherein the dimensions of the collection container are approximately three and one eighth inches wide and one and one half inches deep.

26. A method of collecting a waste sample from a person comprising the steps of:
obtaining the sample collection assembly of claim 13;
removing the cap from the container;
assembling the funnel by pulling the sheet out of the container and spreading out the sheet;
using the second attachment mechanism to secure the outer edge of the sheet to the toilet seat or bedpan;
collect the waste sample;
remove the funnel; and
secure the cap on the container.

27. A method of collecting a waste sample from a person comprising the steps of:
obtaining the sample collection assembly of claim 17;
removing the cap from the container;
assembling the funnel by pulling the first sheet and the second sheet out of the container and spreading out the first sheet and the second sheet;
using the second attachment mechanism to secure the outer edge of the first sheet and the outer edge of the second sheet to the toilet seat or bedpan;
collect the waste sample;
remove the funnel; and
secure the cap on the container.
